(19) United States
(12) Patent Application Publication
(10) Pub. No.: US 2023/0310394 A1
(43) Pub. Date: Oct. 5, 2023

(54) SORAFENIB, REGORAFENIB AND NOVEL USE OF ANALOGUE OR DERIVATIVE THEREOF

(71) Applicant: THE SECOND XIANGYA HOSPITAL OF CENTRAL SOUTH UNIVERSITY, Changsha, Hunan (CN)

(72) Inventors: Tuo DENG, Changsha, Hunan (CN); Wanyu HU, Changsha, Hunan (CN); Ai ni WU FU ER, Changsha, Hunan (CN)

(73) Assignee: THE SECOND XIANGYA HOSPITAL OF CENTRAL SOUTH UNIVERSITY, Changsha, Hunan (CN)

(21) Appl. No.: 18/016,130

(22) PCT Filed: Jun. 28, 2021
(86) PCT No.: PCT/CN2021/102607

§ 371 Date: Jan. 13, 2023

(30) Foreign Application Priority Data
Jul. 13, 2020 (CN) 202010668274.3

(51) Int. Cl. A61K 31/44; A61P 35/00

(52) U.S. Cl. CPC A61K 31/44; A61P 35/00

(57) ABSTRACT

A use of sorafenib or regorafenib or an analogue or a derivative thereof in the preparation of pharmaceuticals for treatment of myeloproliferative neoplasms. The myeloproliferative neoplasms are polycythemia vera or myeloproliferative neoplasms having drug resistance.

### CROSS REFERENCE TO RELATED APPLICATIONS

This application claims the priority of Chinese Patent Application No. 202010668274.3, filed with the China National Intellectual Property Administration on Jul. 13, 2020, and titled with “SORAFENIB, REGORAFENIB AND NOVEL USE OF ANALOGUE OR DERIVATIVE THEREOF”, which is hereby incorporated by reference.

### FIELD

The present disclosure relates to the field of medicine, and in particular to a novel use of sorafenib, regorafenib and an analog or a derivative thereof, especially a use of sorafenib, regorafenib and an analog or a derivative thereof in the preparation of a medicament for treatment of polycythemia vera or myeloproliferative neoplasms having ruxolitinib resistance.

### BACKGROUND

Myeloproliferative neoplasms (MPNs) refer to a group of neoplastic diseases caused by the clonal proliferation of relatively mature one-lineage or multi-lineage myeloid cells. The 2016 World Health Organization (WHO) classification of myeloid neoplasms revision includes the following myeloproliferative neoplasms (MPNs): chronic myeloid leukemia (CML), BCR-ABL¹⁺; chronic neutrophilic leukemia; polycythemia vera (PV); primary myelofibrosis (PMF); essential thrombocythemia (ET); chronic eosinophilic leukemia (not otherwise specified); and unclassifiable MPN. PV, ET, and PMF are classified as philadelphia-negative classical MPNs, which clinically manifest as one or more blood cells hyperplasia with hepatomegaly, splenomegaly, or lymphadenopathy. MPNS is a clonal hematopoietic stem cell disease, in which the main gene mutations that drive the disease include JAK2/V617F, CALR, and MPL mutations. Among them, JAK2/V617F mutation is the most common type, which can be found in 95% of PV, 50-60% of ET and 55-65% of PMF patients. Insertion or deletion mutations in exon 12 of JAK2 can be found in approximately 3% of PV patients.

The goal of treatment in patients with PV and ET is to avoid thrombotic and bleeding complications. The main goal of PMF treatment is to prolong survival, and if conditions permit, allogeneic stem-cell transplantation (AlloSCT) should be used to achieve cure as much as possible. If prolonging survival or cure cannot be achieved, the main goal of treatment is changed to relieve symptoms and improve quality of life. Currently, MPNs are clinically diagnosed according to the “2016 World Health Organization Classification of Myeloid Tumors and Acute Leukemia”, through which risk stratification and corresponding treatment measures are conducted.

Ruxolitinib (RUX), as a JAK1/JAK2 inhibitor, is approved by the FDA as a first-line treatment for intermediate and high-risk myelofibrosis (MF) including primary myelofibrosis, post-polycythemia vera myelofibrosis and post-essential thrombocythemia myelofibrosis. It is used as a second-line drug for PV patients with intolerance and resistance to hydroxyurea (HU). Phase II and phase III clinical trial results suggest that compared with the best available therapy (BAT), RUX can reduce spleen volume and alleviate symptoms in patients with intermediate and high-risk MF and PV, while RUX treatment showed no effect on ET patients with intolerance and resistance to HU. Results of the COMFORT and RESPONSE clinical trials show that anemia and thrombocytopenia were dose-dependent toxic side effects of RUX in the treatment of patients with MF and PV Patients with MF treated with RUX had more severe anemia, of which 51% required at least one red blood cell transfusion, and about 5% discontinued treatment for this reason. In addition, long-term use of type I JAK inhibitors such as RUX can induce the occurrence of drug resistance, and cross-resistance among several type I JAK inhibitors has also been found in clinical studies. Type I JAK inhibitors do not significantly reduce mutant allele burden and thus have limited therapeutic potential. In sum, RUX, as a type I JAK2 inhibitor, represents an important improvement in the treatment of MPNs, but it has not achieved high expectations. BMS911543 is a more JAK2-selective inhibitor, whose phase I/II studies (NCT01236352) have been completed, while the phase III studies of other compounds with JAK2/FLT3 inhibitory profiles (such as fedratinib and pacritinib) have not been completed due to adverse events.

Bone marrow transplantation is the only cure for MPNs, but there are still some issues that need to be addressed. The choices of transplantation mode and protocol are uncertain, and whether to choose allogeneic transplantation or haploidentical transplantation is unclear. In addition, transplantation-related mortality and the long-term nature of myeloproliferative neoplasms must be considered when selecting transplantation. At present, bone marrow transplantation is mainly used to treat high-risk myelofibrosis patients, while the timing of bone marrow transplantation for patients with other types of MPNs needs to be further explored and confirmed by research. Bone marrow transplantation is expensive and is not an option for most patients in the current medical environment.

In conclusion, ruxolitinib is a milestone drug in the treatment of MPNs, but the lack of good therapeutic drugs after patients become ruxolitinib resistant is a major challenge in the current treatment of PNs. There are currently no drugs available for ruxolitinib-resistant patients.

### SUMMARY

In view of this, the purpose of the present invention is to provide a drug for myeloproliferative neoplasms, especially for ruxolitinib-resistant patients, targeting the problems existing in the prior art.

Sorafenib is a small molecule compound that inhibits tumor cell proliferation, angiogenesis and increases apoptosis in a wide range of tumor models. As an oral receptor tyrosine kinase inhibitor, it inhibits factors involved in tumorigenesis and tumor progression, such as Raf serine/threonine kinase and receptor tyrosine kinases (vascular endothelial growth factor receptor 1, 2, 3 and platelet-derived growth factor-β, Flt-3 and c-kit). Sorafenib is approved by the US FDA for the treatment of advanced inoperable hepatocellular carcinoma (HCC), advanced renal cell carcinoma (RCC), and advanced radioiodine-refractory differentiated thyroid cancer (RRDTC). Sorafenib has a molecular formula of C₂₁H₁₆ClF₃N₄O₃, a molecular weight of 464.83, and a structural formula as shown in formula I.

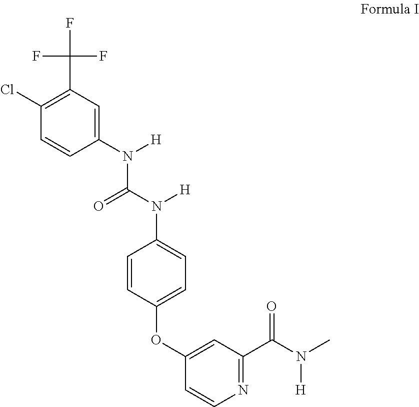 [SMILES: CNC(=O)c1cc(Oc2ccc(NC(=O)Nc3ccc(Cl)c(C(F)(F)F)c3)cc2)ccn1]

The sorafenib analog, regorafenib, has a molecular formula of C₂₁H₁₅ClF₄N₄O₃, a molecular weight of 482.82, and a structural formula as shown in formula II.

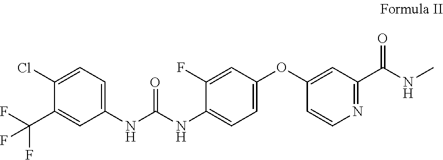 [SMILES: CNC(=O)c1cc(Oc2ccc(NC(=O)Nc3ccc(Cl)c(C(F)(F)F)c3)c(F)c2)ccn1]

In the present invention, the inhibitory effect of sorafenib on myeloproliferative neoplasms cells (drug-resistant and non-drug-resistant) is clarified by cell line models.

In some embodiments of the present invention, two common drug-resistant cell models HEL^{PE} and HEL^{RE} are constructed based on HEL cell (Human erythroleukemia cell line), which is a commonly used human myeloproliferative tumor cell line containing JAK2-V617F mutation. The HEL^{PE} model is namely the HEL-persistent model, which is constructed by treating HEL-naive cells with a high concentration of ruxolitinib that exceeds the IC₅₀ concentration of naive cells by more than 100 times. The culture medium of cells is changed every two days, the cells that cannot tolerate the high concentration of ruxolitinib die quickly, while the proliferated cells are DTP (drug-tolerant-persisters), and this cell subset is difficult to be killed by antitumor drugs. Another drug resistance model is HELE, namely the HEL-resistant model, which is constructed by treating naive cells with ruxolitinib from a starting concentration lower than the IC₅₀ concentration of naive cells, slowly increasing to a high concentration to keep the cells from being killed.

In some embodiments of the present invention, two drug-resistant cell lines are treated with increasing concentrations of sorafenib, and the cell proliferation is detected using CellTiter-Lumi™ luminescence method. The results show that sorafenib can successfully inhibit the proliferation of cells in the HEL^{PE} model and the HEL^{RE} model.

In some embodiments of the present invention, two drug-resistant cell lines are treated with increasing concentrations of sorafenib, and the apoptosis of cells is detected by flow cytometry after Annexin V-PI staining. The results show that sorafenib can promote the apoptosis of cells in HEL^{PE} model and HELE model.

In some embodiments of the present invention, HEL^{RE}-resistant cell line is treated with increasing concentrations of regorafenib (a sorafenib analog), of which the cell proliferation is detected using CellTiter-Lumi™ luminescence method, and the cell apoptosis is detected by flow cytometry after Annexin V-PI staining. The results show that regorafenib can successfully inhibit the proliferation of cells in the HEL^{RE} model and promote the apoptosis of the cells in the HELE model.

It can be seen that sorafenib, regorafenib and an analog or a derivative thereof can be used for the treatment of ruxolitinib-resistant MPN diseases.

Further, in some embodiments of the present invention, HEL, a human cell model commonly used in the study of PV, is treated with increasing concentrations of sorafenib, regorafenib and an analog or a derivative thereof, of which the cell proliferation is detected using CellTiter-Lumi™ luminescence method. The results show that sorafenib, regorafenib and an analog or a derivative thereof can successfully inhibit the proliferation of HEL cells. Therefore, the present invention provides use of sorafenib, regorafenib and an analog or a derivative thereof in the preparation of a medicament for inhibiting the proliferation of HEL cells.

In some embodiments of the present invention, HEL, a human cell model commonly used in the study of PV, is treated with increasing concentrations of sorafenib, regorafenib and an analog or a derivative thereof, of which the cell apoptosis is detected by flow cytometry after Annexin V-PI staining. The results show that sorafenib, regorafenib and an analog or a derivative thereof can promote the apoptosis of HEL cells. Therefore, the present invention provides use of sorafenib, regorafenib and an analog or a derivative thereof in the preparation of a medicament for promoting the apoptosis of HEL cells.

It can be seen that sorafenib, regorafenib and an analog or a derivative thereof can be used to treat myeloproliferative neoplasms, especially polycythemia vera, by inhibiting the proliferation of HEL cells and promoting the apoptosis of HEL cells.

To sum up, the present invention provides use of sorafenib, regorafenib and an analog or a derivative thereof in the preparation of a medicament for the treatment of myeloproliferative neoplasms.

Further, the myeloproliferative neoplasms are polycythemia vera or myeloproliferative neoplasms having drug resistance.

In some embodiments, the myeloproliferative neoplasms having drug resistance are myeloproliferative neoplasms having ruxolitinib resistance.

In some embodiments, the myeloproliferative neoplasms having drug resistance are polycythemia vera having drug resistance, primary myelofibrosis having drug resistance, and essential thrombocythemia having drug resistance.

Among them, the drug includes sorafenib, regorafenib and an analog or a derivative thereof.

The drug includes an analog of sorafenib: regorafenib, which differs from sorafenib in that one H in sorafenib becomes F in regorafenib. Regorafenib is an oral tyrosine kinase inhibitor approved for the treatment of refractory metastatic colorectal cancer, advanced gastrointestinal stromal tumors previously treated with imatinib and sunitinib, and unresectable hepatocellular carcinoma that progressed after use of sorafenib.

In some specific embodiments of the present invention, the sorafenib used is specifically sorafenib mesylate.

Further, the medicament also comprises a pharmaceutically acceptable excipient.

The medicament can be in any dosage form in the current pharmaceutical field, including an oral preparation or an injection preparation.

Each drug dosage form can be prepared by selecting appropriate acceptable excipients according to the actual needs of the dosage form, which belongs to the conventional dosage form preparation technology in the art. For example, capsules, tablets, injection powder and so on.

As can be seen from the above technical solutions, the present invention provides use of sorafenib, regorafenib and an analog or a derivative thereof in the preparation of a medicament for the treatment of myeloproliferative neoplasms. The myeloproliferative neoplasms are polycythemia vera or myeloproliferative neoplasms having ruxolitinib resistance. The use of sorafenib, regorafenib and an analog or a derivative thereof for the treatment of polycythemia vera provides a new treatment approach for the majority of patients with polycythemia vera, and provides more choices for clinicians and patients. For patients with myeloproliferative neoplasms having ruxolitinib resistance, sorafenib, regorafenib and an analog or a derivative thereof can provide patients with continued oral drug therapy so as to avoid bone marrow transplantation. Sorafenib, regorafenib and an analog or a derivative thereof can be chemically synthesized, and the cost thereof is lower than that of biological preparations. They have been approved by FDA and NMPA for marketing and clinical treatment. They have mild side effects, which are well tolerated by clinical patients, and have lower financial burden.

### BRIEF DESCRIPTION OF DRAWINGS

In order to illustrate the examples of the present invention or the technical solutions in the prior art more clearly, the drawings that are required in the description of the examples or the prior art will be introduced briefly in the following.

FIG. 1 is the result diagram of the construction of the drug-resistant cell models HEL^{PE} and HEL^{RE} in Example 1, wherein a is the result diagram of the successful construction of the HEL-persistent model; b is the result diagram of the successful construction of the HEL-resistant model;

FIG. 2 is the result diagram of the cell proliferation of two drug-resistant cell lines treated with sorafenib in Example 2 detected by CellTiter-Lumi™ luminescence method, wherein a is the cell proliferation diagram of HEL-persistent model; b is the cell proliferation diagram of HEL-resistant model;

FIG. 3 is the result diagram of the apoptosis of two drug-resistant cell lines treated with sorafenib in Example 3 detected by flow cytometry after Annexin V-PI staining, wherein a is the result diagram of the apoptosis of HEL-persistent model, and b is the result diagram of the apoptosis of HEL-resistance model;

FIG. 4 is the result diagram of the cell proliferation of PV cell line treated with sorafenib in Example 4 detected by CellTiter-Lumi™ luminescence method;

FIG. 5 is the result diagram of the apoptosis of PV cell line treated with sorafenib in Example 5 detected by flow cytometry after Annexin V-PI staining;

FIG. 6 is the result diagram of the proliferation of HEL-resistant model cells treated with regorafenib in Example 6 detected by CellTiter-Lumi™ luminescence method;

FIG. 7 is the result diagram of the apoptosis of HEL-resistant model cells treated with regorafenib in Example 7 detected by flow cytometry after Annexin V-PI staining;

FIG. 8 is the result diagram of the cell proliferation of PV cell line treated with regorafenib in Example 8 detected by CellTiter-Lumi™ luminescence method;

FIG. 9 is the result diagram of the apoptosis of PV cell line treated with regorafenib in Example 9 detected by flow cytometry after Annexin V-PI staining.

### DETAILED DESCRIPTION

The present invention discloses new use of sorafenib, regorafenib and an analog or a derivative thereof. Those skilled in the art can learn from the content of this document and appropriately improve the process parameters to achieve the present invention. It should be particularly noted that all similar substitutions and modifications are obvious to those skilled in the art, and they are deemed to be included in the present invention. The method and use of the present invention have been described through the preferred embodiments, and it is obvious that those skilled in the art can make modifications or appropriate changes and combinations to the method and use described herein without departing from the content, spirit and scope of the present invention to achieve and apply the technology of the present invention.

In order to further understand the present invention, the technical solutions in the examples of the present invention will be clearly and completely described below with reference to the examples of the present invention. Apparently, the described examples are only a part of the examples of the present invention, rather than all the examples. Based on the examples of the present invention, all other examples obtained by those of ordinary skill in the art without creative efforts shall fall within the protection scope of the present invention.

Unless otherwise specified, the reagents involved in the examples of the present invention are all commercially available products, which can be purchased through commercial channels.

### Example 1. Construction of Two Common Drug-Resistant Cell Models (HEL^{PE}, HEL^{RE})

I. Materials and methods

1. Cell Line

HEL (Human erythroleukemia cell line) and drug-resistant HEL cells were cultured in RPMI medium (Gibco) containing 20% heat-inactivated fetal bovine serum (Gibco) and 1% penicillin/streptomycin (Gibco).

HEL^{PE} model, namely HEL-persistent model, was constructed by treating HEL blast cells with a high concentration of ruxolitinib that exceeded the IC₅₀ concentration of blast cells by more than 100 times, wherein the concentration of ruxolitinib used herein was 2.0 μM. The culture medium of the cells was changed every two days, the cells that cannot tolerate the high concentration of ruxolitinib died quickly, while the proliferated cells were DTP (drug-tolerant-persisters), and this cell subset was difficult to be killed by antitumor drugs. Stable drug-resistant cells were obtained after 4-6 weeks. Another drug resistance model is HEL^{RE}, namely the HEL-resistant model, which was constructed by treating blast cells with ruxolitinib from a starting concentration lower than the IC₅₀ concentration of blast cells, slowly increasing to a high concentration to keep the cells from being killed. The starting concentration used herein was 0.1 μM, the drug was added as soon as the cells proliferated, the dosing gradient was 1.25-fold increments, and the final concentration was 2.0 μM. Stable drug-resistant cells were obtained after 4-6 weeks.

2. Inhibitors

Ruxolitinib, sorafenib and regorafenib were all purchased from Selleck or Bayer, and dissolved in DMSO to prepare stock solutions at a concentration of 10 mM, and the stock solutions thereof were frozen at −80° C., which were diluted with RPMI medium to the specified folds as working solutions to treat cells. Sorafenib was specifically sorafenib mesylate.

3. In Vitro Inhibition Test

To test the anti-proliferative effect of the inhibitors, the above cell lines were cultured at a 3000 cells/200 μL per well, added with increasing concentrations of the inhibitors (concentration gradient: 0, 1, 2.5, 5, 10, 20 μM), then supplemented with DMSO to an equal volume. 4 parallel replicate groups and 3 blank wells (culture medium wells without cells) were set. After 72 hours, the cell proliferation was detected by a multi-plate reader using CellTiter-Lumi™ luminescence method (Beyotime), and the IC₅₀ was calculated by Graph Pad prism.

The calculation formula of cell proliferation rate: cell proliferation rate=(Luminescence value of drug-treated group—average Luminescence value of blank wells)/(Luminescence value of DMSO control group—average Luminescence value of blank wells)×100%.

The method to evaluate whether the model is successfully constructed is to compare the IC₅₀ of drug-resistant cells and naive cells, the obtained ratio was the drug-resistance index, and if the ratio was greater than 3, then the model was successfully constructed.

II. Analysis of the Results

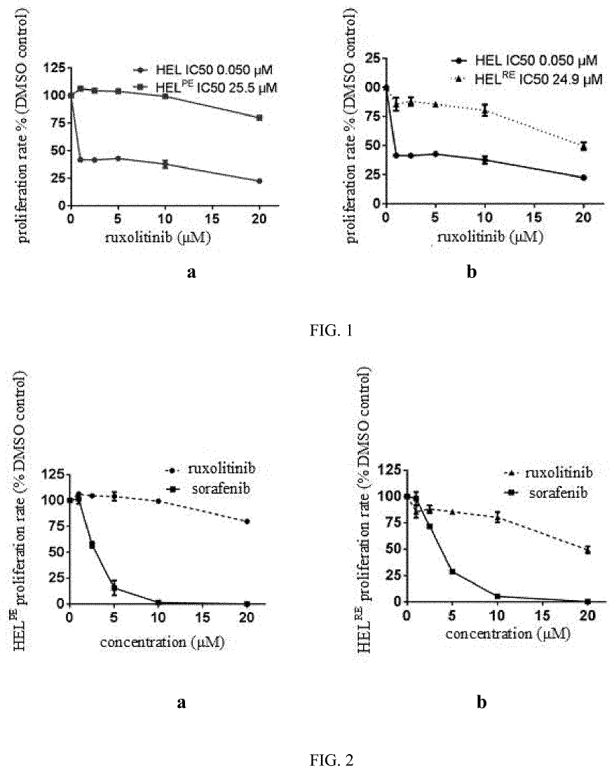

In FIG. 1, a shows that the IC₅₀ of HEL cells was 0.050 (0.002-1.24) μM, the IC₅₀ of HEL^{PE} was 25.5 (137-47.5) μM, and the drug-resistance index thereof was 510, indicating the successful construction of the HEL-persistent model; b shows that the IC₅₀ of HELE was 24.9 (15.6-39.7) μM, and the drug-resistance index thereof was 498, indicating the successful construction of the HEL-resistant model. The results of the proliferation rate in the figure are expressed as the mean±standard deviation, and the IC₅₀ is expressed as the mean. Results are shown as means (95% confidence interval) in the analysis.

### Example 2. Sorafenib can Inhibit the Proliferation of Two Drug-Resistant Cell Lines

Method: The drug-resistant cell lines were treated with increasing concentrations of sorafenib, and the cell proliferation was detected by CellTiter-Lumi™ luminescence method, wherein the method was the same as that in Example 1, and the results are shown in FIG. 2.

Result: FIG. 2a reflects that in the ruxolitinib-resistant cell model HEL-persistent, the IC₅₀ value of sorafenib was 2.80 μM, which was 1/9 of the IC₅₀ value of ruxolitinib of 25.5 μM, and FIG. 2b reflects that in the ruxolitinib-resistant cell model HEL-resistant, the IC₅₀ value of sorafenib was 3.56 μM, which was 1/7 of the IC₅₀ value of ruxolitinib of 24.9 μM. This indicates that sorafenib can inhibit the proliferation of ruxolitinib-resistant cells, and this effect is enhanced with the increase of drug concentration.

### Example 3. Sorafenib can Promote the Apoptosis of Two Drug-Resistant Cell Lines

I. Materials and Methods

1. Cell Lines and Inhibitors were the Same as Those in Example 1.

2. Apoptosis Detection

To test the pro-apoptotic effect of the inhibitor, the drug-resistant cell lines were treated with increasing concentrations of sorafenib for 24 hours (concentrations: 0, 2.5, 5, 10 μM), and supplemented with DMSO to an equal amount. Three parallel replicate groups were set up, and the apoptosis of cells was detected by flow cytometry after Annexin V-PI staining.

The calculation formula of the apoptosis rate: apoptosis rate=ratio of early apoptotic cells (Annexin V+/PI−)+ratio of late apoptotic cells and necrotic cells (Annexin V+/PI+).

II. Analysis of the Results

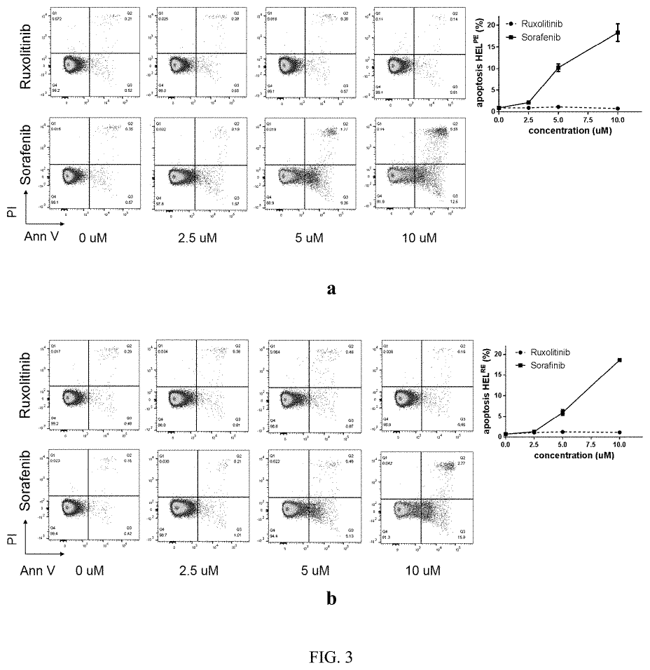

In FIG. 3a, the drug concentrations (average apoptosis rate) of the ruxolitinib-treated group were: 0 μM (0.91%), 2.5 μM (0.920%), 5 μM (1.11%), 10 μM (0.740%); the drug concentrations (apoptosis rate) of the sorafenib-treated group were 0 μM (0.910%), 2.5 μM (2.16%), 5 μM (11.1%), and 10 μM (18.0%). In FIG. 3b, the drug concentrations (apoptosis rate) of the ruxolitinib-treated group were 0 μM (0.760%), 2.5 μM (1.16%), 5 μM (1.35%), and 10 μM (1.05%); the drug concentrations (average apoptosis rate) of the sorafenib-treated group were 0 μM (0.760%), 2.5 μM (1.22%), 5 μM (5.58%), and 10 μM (18.6%). This suggests that sorafenib can promote apoptosis of the HEL^{PE}/RE model, and this effect is enhanced with the increase of drug concentration.

### Example 4. Sorafenib can Inhibit the Proliferation of PV Cell Line

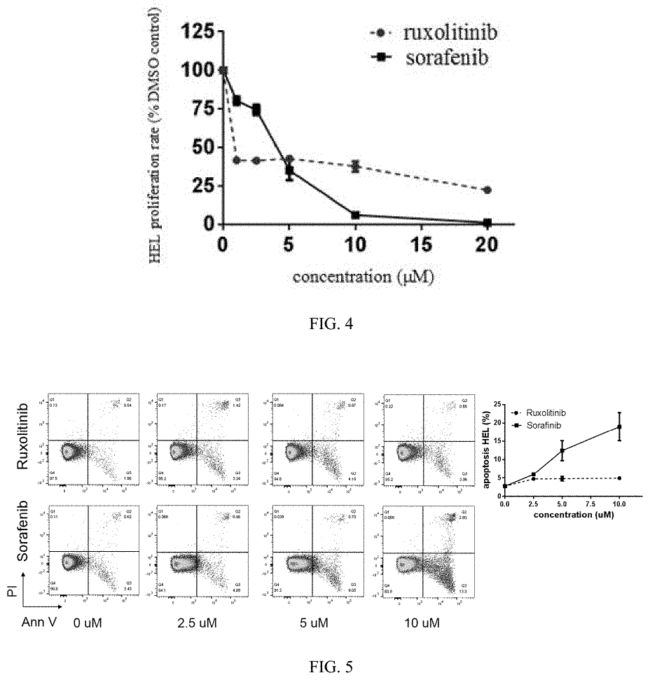

Method: The blast cell line was treated with increasing concentrations of sorafenib, and the cell proliferation was detected by CellTiter-Lumi™ luminescence method, wherein the method was the same as that in Example 1, and the results are shown in FIG. 4.

In FIG. 4, the drug concentrations (average proliferation rate) of the ruxolitinib-treated group were: 0 μM (100%), 1 μM (41.4%), 2.5 μM (40.6%), 5 μM (42.8%), 10 μM (40.8%), and M (22.9%); the drug concentrations (average proliferation rate) of the sorafenib-treated group were: 0 μM (100%), 1 μM (80.3%), 2.5 μM (72.1%), 5 μM (40.0%), 10 μM (6.35%), and 20 μM (0.828%). This suggests that sorafenib can inhibit the proliferation of human PV cell line-HEL cells, this effect is enhanced with the increase of drug concentration, and the inhibitory effect of sorafenib is better than that of ruxolitinib at the concentration of 5 μM and above.

### Example 5. Sorafenib can Promote the Apoptosis of PV Cell Line

Method: The blast cell line was treated with increasing concentrations of sorafenib, and the apoptosis of cells was detected by flow cytometry after Annexin V-PI staining, wherein the method was the same as that in Example 3, and the results are shown in FIG. 5.

In FIG. 5, the drug concentrations (average apoptosis rate) of the ruxolitinib-treated group were: 0 μM (2.88%), 2.5 μM (4.65%), 5 μM (5.07%), and 10 μM (4.59%); the drug concentrations (apoptosis rate) of the sorafenib-treated group were 0 μM (2.88%), 2.5 μM (5.82%), 5 μM (10.9%), and 10 μM (16.1%). This indicates that sorafenib can promote the apoptosis of human PV cell line-HEL cells, this effect is enhanced with the increase of drug concentration, and the pro-apoptotic effect of sorafenib is better than that of ruxolitinib at the concentration of 2.5 μM and above.

### Example 6. Regorafenib can Inhibit the Proliferation of HEL-Resistant Cells

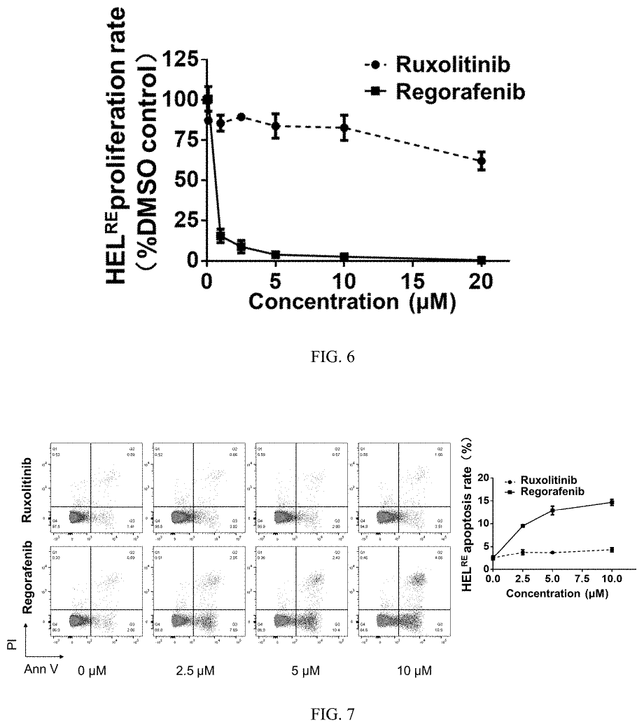

Method: The drug-resistant cell line was treated with increasing concentrations of regorafenib, and the cell proliferation was detected by CellTiter-Lumi™ luminescence method, wherein the method was the same as that in Example 1, and the results are shown in FIG. 6.

FIG. 6 reflects that in the ruxolitinib-resistant cell model HEL-resistant, the IC₅₀ value of regorafenib was 0.514 μM, which was 1/290 of the ruxolitinib IC₅₀ value of 149 μM. This indicates that regorafenib can inhibit the proliferation of ruxolitinib-resistant cells, and this effect is enhanced with the increase of drug concentration.

### Example 7. Regorafenib can Promote the Apoptosis of HEL-Resistant Cells

Methods: The drug-resistant cell line was treated with increasing concentrations of regorafenib, and the apoptosis of cells was detected by flow cytometry after Annexin V-PI staining, wherein the method was the same as that in Example 3, and the results are shown in FIG. 7.

In FIG. 7, the drug concentrations (average apoptosis rate) of the ruxolitinib-treated group were: 0 μM (2.51%), 2.5 μM (3.75%), 5 μM (3.70%), and 10 μM (4.34%); the drug concentrations (apoptosis rate) of the regorafenib-treated group were 0 μM (2.51%), 2.5 μM (9.50%), 5 μM (12.9%), and 10 μM (14.7%). This indicates that regorafenib can promote the apoptosis of ruxolitinib-resistant cells, and this effect is enhanced with the increase of drug concentration.

### Example 8. Regorafenib can Inhibit the Proliferation of PV Cell Line

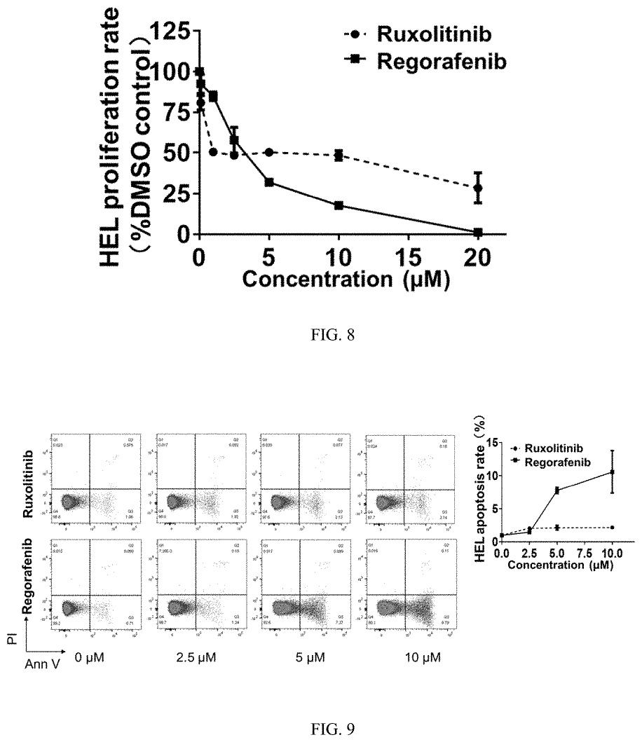

Method: The blast cell line was treated with increasing concentrations of regorafenib, and the cell proliferation was detected by CellTiter-Lumi™ luminescence method, wherein the method was the same as that in Example 1, and the results are shown in FIG. 8.

In FIG. 4, the drug concentrations (average proliferation rate) of the ruxolitinib-treated group were: 0 μM (100%), 1 μM (50.5%), 2.5 μM (48.4%), 5 μM (50.2%), 10 μM (48.4%), and 20 μM (28.5%); the drug concentrations (average proliferation rate) of the regorafenib-treated group were: 0 μM (100%), 1 μM (84.7%), 2.5 μM (57.7%), 5 μM (32.1%), 10 μM (17.7%), and 20 μM (1.15%). This suggests that regorafenib can inhibit the proliferation of human PV cell line-HEL cells, and this effect is enhanced with the increase of drug concentration.

### Example 9. Regorafenib can Promote the Apoptosis of PV Cell Line

Methods: The blast cell line was treated with increasing concentrations of regorafenib, and the apoptosis of cells was detected by flow cytometry after Annexin V-PI staining, wherein the method was the same as that in Example 3, and the results are shown in FIG. 9.

In FIG. 5, the drug concentrations (average apoptosis rate) of the ruxolitinib-treated group were: 0 μM (0.965%), 2.5 μM (2.05%), 5 μM (2.14%), and 10 μM (2.16%); the drug concentrations (apoptosis rate) of the regorafenib-treated group were 0 μM (0.965%), 2.5 μM (4.31%), 5 μM (7.76%), and 10 μM (10.6%). This indicates that regorafenib can promote the apoptosis of human PV cell line-HEL cells, and this effect is enhanced with the increase of drug concentration.

The new use of sorafenib, regorafenib and an analog or a derivative thereof provided by the present invention have been introduced in detail above. The principles and implementations of the present invention are described herein by using specific examples, and the descriptions of the above examples are only used to help understand the method and the core idea of the present invention. It should be noted that for those skilled in the art, several improvements and modifications can also be made to the present invention without departing from the principle of the present invention, and these improvements and modifications also fall within the protection scope of the claims of the present invention.

1. A method of treating myeloproliferative neoplasms, comprising administering sorafenib, regorafenib, an analog or a derivative thereof to a subject in need thereof.

2. The method according to claim 1, wherein the myeloproliferative neoplasms are polycythemia vera or myeloproliferative neoplasms having drug resistance.

3. The method according to claim 2, wherein the myeloproliferative neoplasms having drug resistance are myeloproliferative neoplasms having ruxolitinib resistance.

4. The method according to claim 2, wherein the myeloproliferative neoplasms having drug resistance is selected from the group consisting of polycythemia vera having drug resistance, primary myelofibrosis having drug resistance, and essential thrombocythemia having drug resistance.

5. A method of inhibiting the proliferation of HEL cells, comprising administering sorafenib, regorafenib, an analog or a derivative thereof to a subject in need thereof.

6. A method of promoting apoptosis of HEL cells, comprising administering sorafenib, regorafenib, an analog or a derivative thereof to a subject in need thereof.

7. The method according to claim 1, wherein sorafenib, regorafenib, an analog or a derivative thereof is administered in the form of a medicament, and the medicament comprises a pharmaceutically acceptable excipient.

8. The method according to claim 1, wherein sorafenib, regorafenib, an analog or a derivative thereof is administered in the form of a medicament, and the medicament is an oral preparation or an injection preparation.
